(19) United States
(12) Patent Application Publication
(10) Pub. No.: US 2026/0345565 A1
(43) Pub. Date: Nov. 5, 2026

(54) COMPOSITIONS AND METHODS FOR PREVENTION OF COGNITIVE DECLINE CAUSED BY DEGENERATIVE DISEASES

(71) Applicant: Anavex Life Sciences Corp., New York, NY (US)

(72) Inventor: Christopher U. Missling, New York, NY (US)

(21) Appl. No.: 19/099,386

(22) PCT Filed: Jul. 28, 2023
(86) PCT No.: PCT/US2023/071251

§ 371 Date: Jan. 28, 2025

Related U.S. Application Data
(60) Provisional application No. 63/393,555, filed on Jul. 29, 2022.

(51) Int. Cl. A61K 31/438; A61P 25/28

(52) U.S. Cl. CPC A61K 31/438; A61P 25/28

(57) ABSTRACT

The present disclosure provides methods, compositions and kits for prophylactic treating or preventing a degenerative disease related to amyloid. The compositions and kits comprise Sigma-1 receptor agonist, an allosteric sigma agonist, and/or a dual agonist of Sigma-1 and M1. The compositions and kits elicit effects of delaying the onset, deterring the progress, and/or diminish the likelihood of the degenerative disease, such as deterring the memory loss and/or cognition decline in Alzheimer's disease.

### CROSS REFERENCE TO RELATED APPLICATIONS

This application claims the benefit of U.S. Provisional Patent Application No. 63/393,555 filed Jul. 29, 2022, entitled “COMPOSITIONS AND METHODS FOR PREVENTION OF COGNITIVE DECLINE CAUSED BY DEGENERATIVE DISEASES”, the entire disclosure of which is incorporated herein by reference.

### FIELD OF THE INVENTION

The present invention relates generally to disease prevention and treatment, such as degenerative diseases involving abnormal amyloid formation and/or deposit.

### BACKGROUND OF THE INVENTION

Neurodegenerative diseases have impeded greatly on patients' daily life, due to memory loss and/or cognition decline. It is critical to develop preventive and prophylactic therapy to block the onset and/or deter the progress of neurodegenerative diseases, with the goal to restore memory and to deter cognition decline.

### SUMMARY OF THE INVENTION

The present disclosure provides methods, compositions and kits for therapeutically or prophylactically treating or preventing a degenerative disease related to amyloid. The compositions and kits comprise a therapeutic agent of sigma-1 receptor agonist, an allosteric sigma agonist, and/or a dual agonist of sigma 1 and M1. The compositions and kits elicit effects of delaying the onset, deterring the progress, and/or diminish the likelihood of the degenerative disease, such as deterring the cognition decline in Alzheimer's disease.

In one aspect of the present disclosure, the therapeutic or the prophylactic agent comprises ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. ANAVEX2-73 (A2-73) has a chemical name of tetrahydro-N, N-dimethyl-2,2-diphenyl-3-furanmethanamine hydrochloride. ANAVEX 19-144 (A19-144) has a chemical name of 1-(2,2-diphenyltetrahydrofuran-3-yl)-N-methylmethanamine hydrochloride. ANAVEX 1-41 (A1-41) has a chemical name of tetrahydro-N, N-dimethyl-5,5-diphenyl-3-furanmethanamine hydrochloride. AV1066 has a chemical name of 1-(3-4(((1R,3S,5S)-adamantan-1-yl)(pheny)methyl)propyl)-4-methylpiperazine. ANAVEX3-71 (A3-71, AF- 710B) has a chemical name of 1-(2,8-dimethyl-1-thia-3,8-diazaspiro[4.5]decan-3-yl)-3-(1H-indol-3-yl)propan-1-one.

In another aspect of the present disclosure, the therapeutic or prophylactic agent comprises A3-71 amorphous form, a A3-71 crystal form, a A3-71 enantiomer, a A3-71 prodrug, or a combination thereof.

In another aspect of the present disclosure, the degenerative disease may comprise Alzheimer's disease, Parkinson's disease, Huntington's disease, Amyotrophic lateral sclerosis, Prion disease, amyloidosis, non-alcoholic fatty liver disease, a disorder related to abnormal amyloid formation, or a disorder related to amyloid deposit.

In another aspect of the present disclosure, the effective amount used is to diminish or reduce the likelihood or seriousness of the degenerative disease. In yet another aspect of the invention, the prophylactively effective amount is the amount to prevent the onset or deter the progress of the degenerative disease. The onset or progress may be manifested by memory loss and/or cognitive decline.

In yet another aspect of the present disclosure, the therapeutically or prophylactically effective amount ranges from about 0.10 mg to about 500 mg, such as between 5 mg and 30 mg. In another aspect, the subject is a human subject or a non-human mammal.

One aspect of the present disclosure encompasses a composition for the aforementioned medical use. Another aspect of the present disclosure encompasses a kit comprising the composition and an instruction for the aforementioned medical use.

In another aspect, the present disclosure provides use of a therapeutically or prophylactically effective amount of a sigma-1 receptor agonist in the manufacture of a medicament for use in any of the methods as disclosed herein.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

FIG. 1 is an illustrative workflow on the rat study of ANAVEX® 3-71 against the onset, development, and/or progression of Alzheimer's Disease. Data are presented as mean±SEM. *P≤0.05; **P≤0.01; ***P≤0.001; ****P≤0.0001.

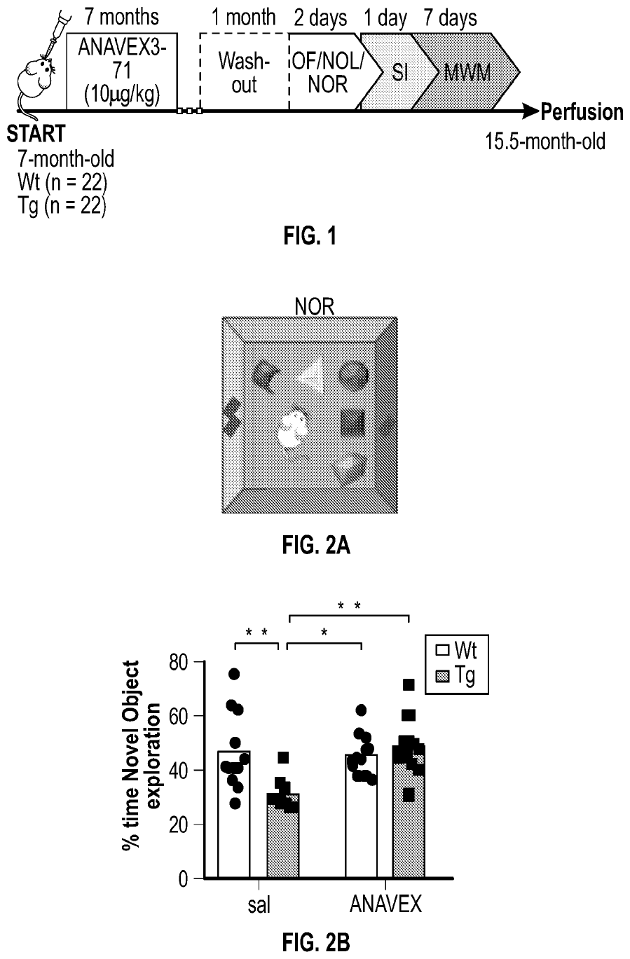

FIG. 2A-2B depicts Novel Object Recognition (NOR) tests and the results. FIG. 2A illustrates rats were exposed to objects. FIG. 2B plots the percentage of rats in each group that were able to find the novel objects: *P≤0.05; **P≤0.01.

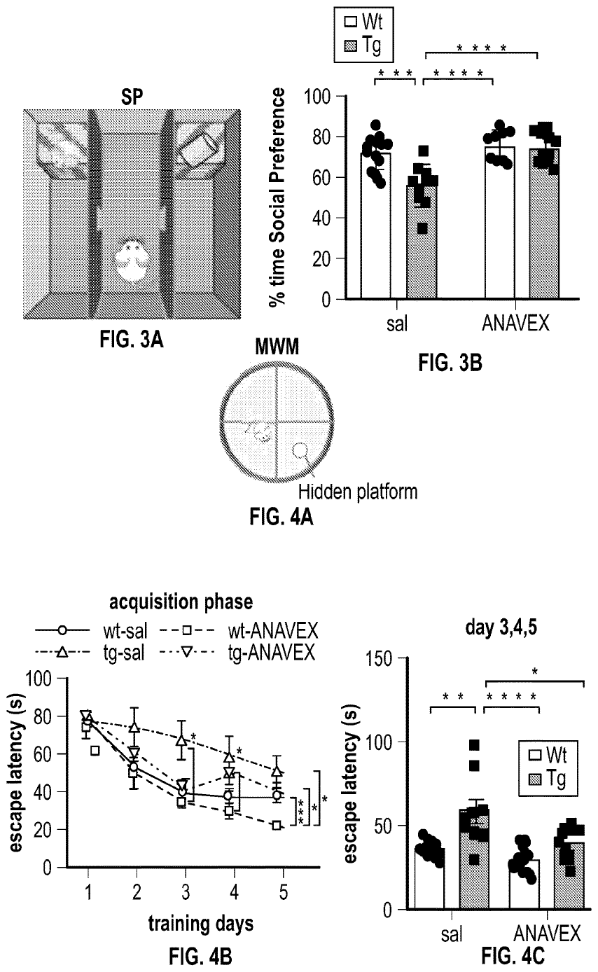

FIG. 3A-3B depicts the Social Preference (SP) test. FIG. 3A illustrates rats were exposed to either social or non-social factor. FIG. 3B plots the percentage of rats in each group that preferred to join the social group: ***P≤0.001; ****P≤0.0001.

FIG. 4A-4C depicts the acquisition phase of the Morris Water Maze (MWM). FIG. 4A illustrates rats were exposed to MWM. FIG. 4B plots the escape latency of each group against the training days. FIG. 4C plots the average escape latencies from day 3 to 5. The Tg-sal required more time to find the hidden platform than wt-sal (P≤0.01). Conversely, Tg-ANAVEX performed significantly better than the Tg-sal (P≤0.05).

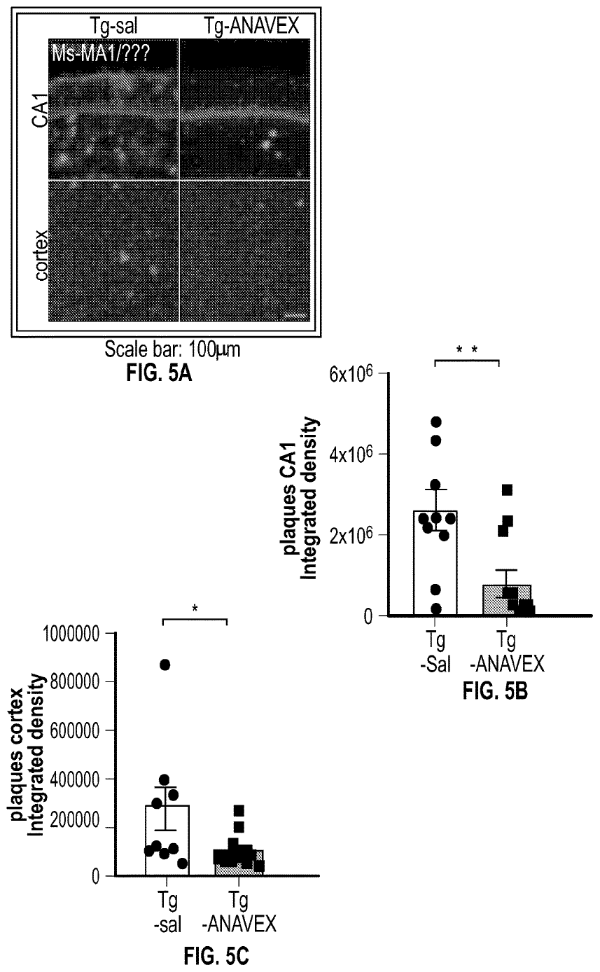

FIG. 5A-5C depicts the impact of A3-71 administration on the cortical and hippocampal extracellular Aβ deposition. FIG. 5A shows the plaques staining in cortex and CA1. FIG. 5B plots the plaques CA1 integrated density cortex in the treated group Tg-ANAVEX and non-treated group Tg-sal, **P≤0.01. FIG. 5C plots the plaques cortex integrated density in the treated group Tg-ANAVEX and non-treated group Tg-sal, *P≤0.05.

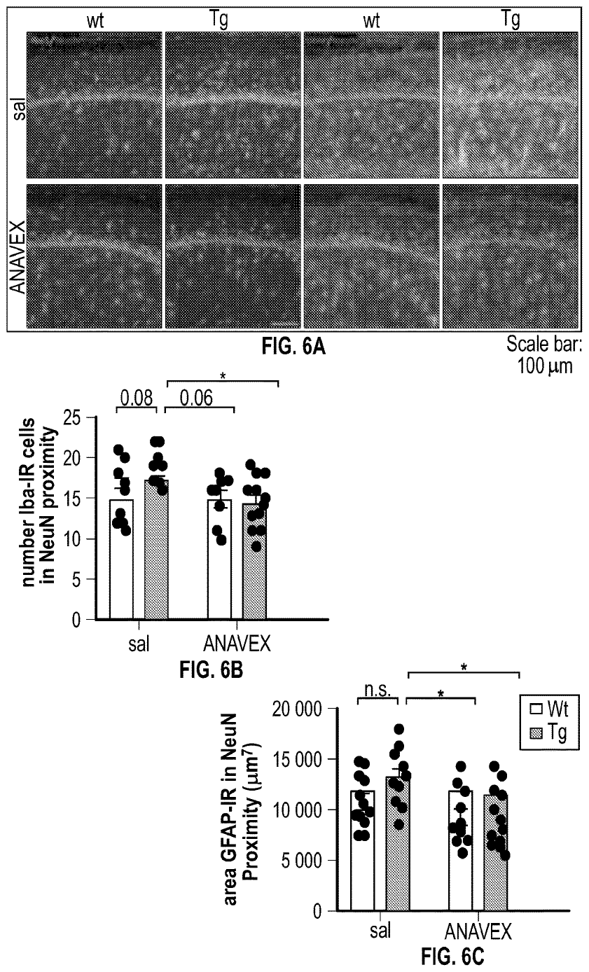

FIG. 6A-6C depicts the impact of A3-71 administration on neurons in hippocampus. FIG. 6A shows the immune staining of different testing groups. FIG. 6B plots the number of Iba1-IR cells in NeuN proximity, *P≤0.05. FIG. 6C plots the number of GFAP-IR cells NeuN proximity, *P≤0.05.

### DETAILED DESCRIPTION

The present disclosure provides methods, compositions and kits for prophylactic treating or preventing a degenerative disease related to amyloid.

Amyloids are aggregates of proteins characterized by a fibrillar morphology of typically 7-13 nm in diameter, a β-sheet secondary structure (known as cross-β) and ability to be stained by particular dyes, such as Congo red. Pathogenic amyloids form when previously healthy proteins lose their normal structure and physiological functions (misfolding) and form fibrous deposits within and around cells. These protein misfolding and deposition processes disrupt the healthy function of tissues and organs, known as amyloidosis. Amyloid plaques are aggregates of misfolded proteins that form in the spaces between nerve cells. When amyloid plaques develop and/or plaques build up in the areas of the brain concerned with memory and other cognitive functions, it may cause a degenerative disease, such as Alzheimer's disease. A degenerative disease is characterized by the worsening condition due to the deterioration of the function and structure of the affected body part, thus causing disability, mortality, and morbidity. One type of degenerative diseases is called neurodegenerative disease (degenerative nervous system diseases), which affects the neurons of the central nervous system, such as Alzheimer's disease, Parkinson's disease, Huntington's disease, Amyotrophic lateral sclerosis, Prion disease, or Multiple sclerosis.

The compositions and kits comprise Sigma-1 receptor agonist, an allosteric sigma agonist, and/or a dual agonist of sigma 1 and muscarinic acetylcholine receptor M1. The compositions and kits elicit effects of preventing or delaying the onset, deterring the progress, and/or diminish the likelihood of the degenerative disease, such as deterring the memory loss and/or cognition decline in Alzheimer's disease. Sigma-1 receptors are shown to be involved in higher-ordered brain functions including memory and cognition. Thus, a Sigma-1 receptor agonist therapy is often prescribed to patients having declined memory or cognition functions, such as those with neurodegenerative disorders. It is discovered that after a subject is being treated with a Sigma-1 receptor agonist, his or her gene expression profiles are altered: a set of selected genes are differentially expressed, and their relevant gene clusters are overrepresented. The altered genetic profile can be used as a benchmark to evaluate the therapeutic effect of another therapeutic agent. It can also be used to select a Sigma-1 receptor agonist for a subject. Further, it can also be used to determine if a subject is responsive to a Sigma-1 receptor agonist therapy. Finally, the Sigma-1 receptor agonist can be used a probe to see if a subject is having, or suspect of having, or at an increased risk of having, a disease linked to the altered gene expression profiles.

The degenerative disease manifested by abnormal amyloid formation and/or deposit may include Alzheimer's disease, Parkinson's disease, Huntington's disease, Amyotrophic Lateral Sclerosis, and Prion disease. Neurodegenerative diseases are a subset of the degenerative diseases, which refer to hereditary and sporadic conditions which are characterized by progressive nervous system dysfunction. These disorders are often associated with atrophy of the central or peripheral nervous system structures and may have symptoms in the form of cognition impairment, memory loss, and movement disorders. Alzheimer's disease is a degenerative disease of the brain characterized by the insidious onset of dementia. Early symptoms include impairment of memory, judgment, attention span, and problem-solving skills. In late stage, severe apraxias and a global loss of cognitive abilities may occur. The disease is marked pathologically by severe cortical atrophy and the triad of senile plaques, neurofibrillary tangles, and neuropil threads. Parkinson's disease is a progressive, degenerative neurologic disease characterized by a tremor that is maximal at rest, retropulsion (i.e. a tendency to fall backwards), rigidity, stooped posture, slowness of voluntary movements, and a masklike facial expression. Pathologic features include loss of melanin containing neurons in the substantia nigra and other pigmented nuclei of the brainstem. Lewy bodies are present in the substantia nigra and locus coeruleus but may also be found in a related condition characterized by dementia in combination with varying degrees of parkinsonism. Secondary Parkinson's disease refer to conditions which feature clinical manifestations resembling primary Parkinson's disease that are caused by a known or suspected condition, such as parkinsonism caused by vascular injury, drugs, trauma, toxin exposure, neoplasms, infections and degenerative or hereditary conditions. Clinical features may include bradykinesia, rigidity, parkinsonian gait, and masked facies. In general, tremor is less prominent in secondary parkinsonism than in the primary form. Huntington's disease is a familial disorder inherited as an autosomal dominant trait and characterized by the onset of progressive chorea and dementia in the fourth or fifth decade of life. Common initial manifestations include paranoia, poor impulse control, depression, hallucinations, and delusions. Late symptoms include intellectual impairment, loss of fine motor control, athetosis, and diffuse chorea involving axial and limb musculature develops, leading to a vegetative state within 10-15 years of disease onset. Its juvenile variant has a more fulminant course including seizures, ataxia, dementia, and chorea. Amyotrophic Lateral Sclerosis is a degenerative disorder affecting upper motor neurons in the brain and lower motor neurons in the brain stem and spinal cord. Disease onset is usually after the age of 50 and the process is usually fatal within 3 to 6 years. Clinical manifestations include progressive weakness, atrophy, fasciculation, hyperreflexia, dysarthria, dysphagia, and eventual paralysis of respiratory function. Pathologic features include the replacement of motor neurons with fibrous astrocytes and atrophy of anterior spinal nerve roots and corticospinal tracts. Prion disease is a group of genetic, infectious, or sporadic degenerative human and animal nervous system disorders associated with abnormal prions. These diseases are characterized by conversion of the normal prion protein to an abnormal configuration via a post-translational process. In humans, these conditions generally feature dementia, ataxia, and a fatal outcome. Pathologic features include a spongiform encephalopathy without evidence of inflammation. Some older literature may occasionally refer to these as unconventional slow virus diseases.

One aspect of the present disclosure encompasses a method of selecting a therapeutic and prophylactic agent for a subject suffering from a disease or a disorder, such as Alzheimer's disease, Parkinson's disease, Huntington's disease, Amyotrophic Lateral Sclerosis, Prion disease, diabetic cardiomyopathy, or non-alcoholic fatty liver disease,

In one aspect of the present disclosure, if the neurodegenerative therapy is identified as ineffective for a subject, a follow-up action can be taken, such as switching to another neurodegenerative therapy, supplementing with another neurodegenerative therapy, adjusting dose if the neurodegenerative therapy being a drug therapy, or switching to a combined neurodegenerative therapy.

In yet another aspect of the present disclosure, the neurodegenerative therapy can be a Sigma-1 receptor agonist therapy, or a NMDA therapy, or a cognition enhancing physical therapy. For example, neurodegenerative therapy can be a drug therapy comprising a Sigma-1 receptor agonist, such as ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. ANAVEX2-73 (A2-73) has a chemical name of tetrahydro-N, N-dimethyl-2,2-diphenyl-3-furanmethanamine hydrochloride.

ANAVEX 19-144 (A19-144) has a chemical name of 1-(2,2-diphenyltetrahydrofuran-3-yl)-N-methylmethanamine hydrochloride. ANAVEX 1-41 (A1-41) has a chemical name of tetrahydro-N, N-dimethyl-5,5-diphenyl-3-furanmethanamine hydrochloride.

AV 1066 has a chemical name of 1-(3-4(((1R,3S,5S)-adamantan-1-yl)(pheny)methyl)propyl)-4-methylpiperazine. ANAVEX3-71 (A3-71, AF-710B) has a chemical name of 1-(2,8-dimethyl-1-thia-3,8-diazaspiro[4.5]decan-3-yl)-3-(1H-indol-3-yl)propan-1-one. For example, the neurodegenerative therapy is a A2-73 drug therapy comprising A2-73 free base, A2-73 amorphous form, A2-73 Crystal Form I, A2-73 Crystal Form II, A2-73 Crystal Form III, (−) A2-73 enantiomer, or (+) A2-73enantiomer.

In another aspect of the present disclosure, the therapeutic agent comprises A3-71 amorphous form, a A3-71 crystal form, a A3-71 enantiomer, a A3-71 prodrug, or a combination thereof.

In another aspect of the present disclosure, the effective amount used is to diminish or reduce the likelihood or seriousness of the degenerative disease. In yet another aspect of the invention, the prophylactically effective amount is the amount to prevent the onset or deter the progress of the degenerative disease. The onset or progress may be manifested by memory loss and/or cognitive decline.

In yet another aspect of the present disclosure, the therapeutically or prophylactically effective amount ranges from about 0.10 mg to about 500 mg, such as between 5 mg and 30 mg of A 2-73. In another aspect, the subject is a human subject or a non-human mammal.

One aspect of the present disclosure encompasses a composition for the aforementioned medical use. Another aspect of the present disclosure encompasses a kit comprising the composition and an instruction for the aforementioned medical uses.

### I. Methods of Treatment

As used herein, the term “treatment” refers an action taken with respect to a subject, which may be a human or an animal subject, intended to ameliorate, prevent, slow, deter, diminish, relieve or cure symptoms and/or effects associated with a recited disease state or condition. A “therapeutic treatment” is a treatment which at least partly prevents, slows, deters, diminishes, relieves or cures symptoms and/or effects associated with a recited disease state or condition.

As used herein to refer to a treatment, the term “prophylactic” refers to action taken with respect to the subject before detection of a symptom or symptomatic condition, to maintain health by delaying, mitigating or avoiding the onset, or diminishing the likelihood or seriousness of a symptom, episode, disease state or condition. A “prophylactic effect” in the context of the present specification should not be understood to encompass total or complete prevention of symptoms or symptomatic conditions.

The term “prophylactically effective amount,” as used herein, pertains to an amount of a compound or composition as disclosed herein, which is effective for producing a desired prophylactic effect when administered as disclosed herein.

The term “therapeutically effective amount,” as used herein, refers to an amount of a compound or composition as disclosed herein, which is effective for producing a desired therapeutic effect when administered as disclosed herein. A therapeutic effect encompasses a prophylactic effect.

### II. Pharmaceutical Compositions

One aspect of the disclosure encompasses a pharmaceutical composition comprising a neurodegenerative agent and/or a Sigma-1 receptor agonist. A pharmaceutical composition comprises a therapeutically or prophylactically effective amount of an active pharmaceutical ingredient, and any pharmaceutically acceptable salt thereof.

Pharmaceutically acceptable salts, include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

When the active pharmaceutical ingredient is A2-73, a composition may comprise from about 1 mg to about 50 g, from about 0.1 to about 5 g, from about 0.5 g to about 3 g, from about 1 mg to about 55 mg, from about 5 mg to about 30 mg, from about 40 mg to about 60 mg, from about 80 mg to about 120 mg, from about 180 mg to about 220 mg, from about 0.1 g to about 5 g, or from about 0.5 g to about 3 g of A 2-73. Formulations comprising A2-73 can be found in, e.g., U.S. U.S. Pat. No. 9,750,746, U.S. Patent Publication No. 20170360798, U.S. Patent Publication No. 20190022052, U.S. Patent Publication No. 20180360796, U.S. Patent Publication No. 20180169059, U.S. Patent Publication No. 20180177756, U.S. Patent Publication No. 20180169060, and U.S. Patent Publication No. 20190117615, the disclosures of which are incorporated herein in their entirety. When the active pharmaceutical ingredient is A3-71, a composition may comprise from about 1 mg to about 50 g, from about 0.1 to about 5 g, from about 0.5 g to about 3 g, from about 1 mg to about 55 mg, from about 5 mg to about 30 mg, from about 40 mg to about 60 mg, from about 80 mg to about 120 mg, from about 180 mg to about 220 mg, from about 0.1g to about 5 g, or from about 0.5 g to about 3 g of A3-71. The active pharmaceutical ingredient may be administered daily, twice daily, or three times daily. The duration of administration may be about 3-6 weeks, 6-11 weeks, 4 months, 5 months, 6 months, 7 months, 8 months, 9 months or chronically. For example, A 2-73 or A3-71 may be administered in an amount of about 40 mg to about 60 mg once daily for about 6-11 weeks, or is administered in about 50 mg daily for up to 11 weeks. A3-71 or A2-73 may also be administered daily in an escalating dose starting from about 10 mg to ending at about 50 mg once daily.

The active pharmaceutical ingredient can be formulated and administered to a subject by any route such as oral, parenteral, intraperitoneal, intravascular, transdermal, subcutaneous, or intrapulmonary in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A pharmaceutical composition also comprises one or more pharmaceutically acceptable excipients. Non-limiting examples of excipients include chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation spray, rectal, or intrapulmonary administration.

The active pharmaceutical ingredient can in general be formulated for improving patient compliance, preventing a subject from removing the drug-delivery device. For instance, Sigma-1 receptor agonists can be formulated for improved patient compliance and preventing removal of a drug-delivery device by providing formulations for extended delivery. Extended delivery can range for periods ranging from more than one day, to months. This may be especially relevant for patients with compromised cognitive and/or motor-control abilities. Extended delivery for periods can range from about 1 day to about 1 year, from about 1 day to about 1 week, from about 3 days to about 1 month, from about 2 weeks to about 6 months, or from about 2 months to about 4 months.

Extended release formulations could be used for substantially continuous delivery of drug at a preselected rate. For example, for A2-73 or A3-71, the drug can be delivered at a rate of from about 1 mg to about 100 mg/day, from about 5 mg to about 30 mg/day, from about 40 to about 60 gm/day, or from about 10 to about 30 gm/day. Appropriate amounts of crystalline A2-73 can be readily determined by the ordinarily skilled artisan based upon, for example, the intended duration of administration of the drug by the extended release formulation, the delivery mechanism, the particular formulation, and the relative potency of the drug among other factors.

### Binders

Non-limiting examples of binders suitable for the formulations of various aspects include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohols, polyethylene glycol, polyols, saccharides, oligosaccharides, polypeptides, oligopeptides, and combinations thereof. The polypeptide may be any arrangement of amino acids ranging from about 100 to about 300,000 Daltons.

The binder can be introduced into the mixture to be granulated in a solid form, including but not limited to a crystal, a particle, a powder, or any other finely divided solid form known in the art. Alternatively, the binder can be dissolved or suspended in a solvent and sprayed onto the mixture in a granulation device as a binder fluid during granulation.

### Diluents

Non-limiting examples of diluents (also referred to as “fillers” or “thinners”) include carbohydrates, inorganic compounds, and biocompatible polymers, such as polyvinylpyrrolidone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

### Disintegrants

Disintegrents can be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

### Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N, N'-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

### Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

### Lubricants and Glidants

The lubricant compositions may be utilized to lubricate ingredients that form a pharmaceutical composition. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The pharmaceutical composition will generally comprise from about 0.01% to about 10% by weight of a lubricant. In some aspects, the pharmaceutical composition will comprise from about 0.1% to about 5% by weight of a lubricant. In a further aspect, the pharmaceutical composition will comprise from about 0.5% to about 2% by weight of a lubricant.

### Dispersants

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants.

### Colorants

Depending upon the aspect of the disclosure, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects of the disclosure.

### Ph Modifiers

Non-limiting examples of pH modifiers include citric acid, acetic acid, tartaric acid, malic acid, fumaric acid, lactic acid, phosphoric acid, sorbic acid, benzoic acid, sodium carbonate and sodium bicarbonate.

### Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

### Antimicrobial Agents

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na2EDTA, and sulfites including but not limited to sulfur dioxide, sodium bisulfite, and potassium hydrogen sulfite.

### Release-Controlling Polymers

Release-controlling polymers may be included in the various aspects of the solid dosage pharmaceutical compositions incorporating compounds according to this disclosure. In one aspect, the release-controlling polymers may be used as a tablet coating. In other aspects, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, arabic gum, carrageens gum, ghafti gum, guar gum, karaya gum, locust bean gum, okra gum, tragacanth gum, scleroglucan gum, xanthan gum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

### Coatings

A solid dosage comprising a compound according to this disclosure may comprise a coating, wherein such a coating may control release of the compound, act as a moisture barrier, or buffer or modify pH. A “control releasing coat” or “controlled release coat” as used herein is defined to mean a functional coat which can for example comprise at least one pH independent polymer, pH dependent polymer (for example enteric or reverse enteric type polymers), soluble polymer, insoluble polymer, lipids, lipidic materials, or combinations thereof. The coating, when applied onto a dosage form, may slow (for example when applied to a normal release matrix dosage form), further slow (for example when applied to a controlled release matrix dosage form) or modify the rate of release of a compound according to this disclosure when applied to an uncoated dosage form. For example, the control releasing coat can be designed such that when the control releasing coat is applied to a dosage form, the dosage form in conjunction with the control releasing coat can exhibit the release of the compound according to this disclosure, such as a “modified-release”, “controlled-release”, “sustained-release”, “extended-release”, “delayed-release”, “prolonged-release,” or combinations thereof. The “control releasing coat” may optionally comprise additional materials that may alter the functionality of the control releasing coat.

The term “moisture barrier” as used herein is one which impedes or retards the absorption of moisture. Compounds according to this disclosure may be hygroscopic and, as such, may be susceptible to decomposition over time under highly humid conditions. The proportion of the components of the moisture barrier and the amount of the moisture barrier optionally applied onto the control-releasing coating or onto the core are typically such that the moisture barrier does not fall within the USP definition and requirement for an enteric coat. Suitably, the moisture barrier may comprise an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier may additionally comprise other conventional inert excipients, which may improve processing of an extended-release formulation.

Coating and matrix materials which may be used in accordance with the invention are those known in the art for use in controlled-release formulations, such as synthetic polymers of the polyvinyl type, e.g., polyvinylchloride, polyvinylacetate and copolymers thereof, polyvinylalcohol, and polyvinylpyrrolidone; synthetic polymers of the polyethylene type, e.g., polyethylene and polystyrene; acrylic acid polymers; biopolymers or modified biopolymers, such as cellulosic polymers, shellac and gelatin; fats, oils, higher fatty acids and higher alcohols (i.e., acids and alcohols containing alkyl chains of at least 10 carbon atoms), for example aluminum monostearate, cetylalcohol, hydrogenated beef tallow, hydrogenated castor oil, 12-hydroxystearl alcohol, glyceryl mono-or dipalmitate; glyceryl mono-, di- or tristearate; myristyl alcohol, stearic acid, stearyl alcohol, and polyethyleneglycols; waxes; sugars and sugar alcohols.

The pH-buffering properties of a coating may be strengthened by introducing into the coating substances chosen from a group of compounds usually used in antacid formulations, for example magnesium oxide, hydroxide or carbonate, aluminum or calcium hydroxide, carbonate or silicate; composite aluminum/magnesium compounds, for example Al2O3·6MgO·CO2·12H2O, (Mg6Al2(OH)16CO3·4H2O), MgO·Al2O3·2SiO2·nH2O, aluminum bicarbonate coprecipitate or similar compounds; or other pharmaceutically acceptable pH-buffering compounds, for example the sodium, potassium, calcium, magnesium and aluminum salts of phosphoric, carbonic, citric or other suitable, weak, inorganic or organic acids; or suitable organic bases, including basic amino acids; and salts or combinations thereof.

A pH-dependent coating serves to release the drug in desired areas of the gastrointestinal (GI) tract, e.g., the stomach or small intestine. When a pH-independent coating is desired, the coating is designed to achieve optimal release regardless of pH-changes in the environmental fluid, e.g., the GI tract. When the coating is formulated to release a compound according to this disclosure in the intestines (especially the upper small intestines), the coating is often called an “enteric coating”. A pH-dependent coating may include, but is not limited to, acrylic acid polymers and copolymers, for example polymers formed from acrylic acid, methacrylic acid, methyl acrylate, ammonio methylacrylate, ethyl acrylate, methyl methacrylate and/or ethyl methacrylate (e.g., Eudragit™); cellulosic polymers such as hydroxypropyl cellulose, hydroxyethyl cellulose, hydroxypropyl methyl cellulose, methyl cellulose, ethyl cellulose, cellulose acetate, cellulose acetate phthalate (CAP), cellulose acetate trimellitate, hydroxypropylmethyl cellulose phthalate, hydroxypropylmethyl cellulose succinate and carboxymethylcellulose sodium; shellac (purified lac); vinyl polymers and copolymers such as polyvinyl pyrrolidone, polyvinyl acetate, polyvinylacetate phthalate (PVAP), vinylacetate crotonic acid copolymer, and ethylene-vinyl acetate copolymers; zein; and salts and combinations thereof.

Oral tables and capsules typically have coatings.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described cells and methods without departing from the scope of the invention, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

As used herein, the term “gene” means a segment of DNA that contains all the information for the regulated biosynthesis of an RNA product, including promoters, exons, introns, and other untranslated regions that control expression. As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function. As used herein, the term “differentially expressed gene” means expression levels of a gene in two experimental conditions or in two samples possess statistically significant difference or change. As used herein, the terms “overrepresented” genes or gene clusters means genes from a pre-defined set are present more than expected.

As used herein, the term “transcriptome analysis” means to characterize transcriptional activity (coding and non-coding), focus on a subset of relevant target genes and transcripts, or profile thousands of genes at once to create a genetic profile. As used herein, the term “mutant” means any heritable variation from the wild-type that is the result of a mutation, e.g., single nucleotide polymorphisms (“SNP”) and insertions/deletions. The term “mutant” is used interchangeably with the terms “marker”, “biomarker”, and “target” throughout the specification.

As used herein, the term “polynucleotide” means any RNA or DNA, which may be unmodified or modified RNA or DNA. Polynucleotides include, without limitation, single-and double-stranded DNA, DNA that is a mixture of single-and double-stranded regions, single-and double-stranded RNA, RNA that is mixture of single-and double-stranded regions, and hybrid molecules comprising DNA and RNA that may be single-stranded or, more typically, double-stranded or a mixture of single-and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art.

Such modifications are well described in basic texts and in more detailed monographs, as well as in a voluminous research literature.

As used herein, the terms “disease”, “disorder” or “dysfunction” are used interchangeably in the present disclosure. They refer to any condition, disorder or disease manifested as one or more physiological, physical and/or psychological symptoms or dysfunctions for which treatment is desirable, and includes previously and newly identified diseases, disorders or dysfunctions on any organs, tissues or biological activities. As used herein, the term “medical use” is any use or means related to restore, remedy, or preserve health or well being of a subject.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The publications discussed above are provided solely for their disclosure before the filing date of the present application. Nothing herein is to be construed as an admission that the invention is not entitled to antedate such disclosure by virtue of prior invention.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Background and Purpose

ANAVEX3-71 (aka “AF710B” or “A3-71”) is a selective allosteric M1 muscarinic and sigma-1 receptor agonist. Acetylcholinesterase inhibitors represent four out of six drugs for treating Alzheimer's disease (AD), with time-limited efficacy likely due to the progressive loss of cholinergic neurons. ANAVEX3-71 takes advantage of the fact that acetylcholine postsynaptic M1 muscarinic brain receptor levels remain unchanged in AD. ANAVEX3-71 treatment attenuated AD hallmarks in McGill-R-Thy1-APP transgenic rats when administered at advanced stages of the AD-like pathology. However, AD therapy in humans has a greater likelihood of success if applied early in the disease prior to extensive brain damage arises. It is desirable to test if administration of ANAVEX3-71 during early amyloid pathology stages could prevent cognitive impairment, plaque deposition, plaque buildup and/or neuroinflammation.

### Example 2. Study Design on Prophylactic Effect of Anavex 3-71 Against Alzheimer's Disease

A rat model study was designed to assess the prophylactic and therapeutic effect of ANAVEX3-71 against onset, development and/or progression of a degenerative disease, such as Alzheimer's Disease. ANAVEX3-71 (10 μg/kg) or saline (sal) was administrated daily p.o. in pre-plaque McGill-R-Thy1-APP (Tg, n=22) and wild-type (Wt, n=22) divided into four groups, for seven months. After one month of drug interruption, behavior tests were performed: Novel Object Recognition (NOR), Morris Water Maze (MWM) and Social Preference (SP). Brains were extracted, fixed and analyzed by histochemistry and immunohistochemistry (see FIG. 1). Statistical analysis was applied to the four groups by 2×2 ANOVA followed by Tukey's multiple comparisons test. For 2-group analysis, Mann Whitney test (data normality violated) was utilized. Data are presented as mean±SEM. *P≤0.05; **P≤0.01; ***P≤0.001; ****P≤P≤0.0001.

### Example 3. Results

As shown in FIG. 2, in the NOR (Novel Object Recognition test), Tg-sal (treatment group with saline administration) explored the novel object significantly less than wt-sal (P≤0.01, wild type with saline administration). The impairment was rescued in Treatment Group with A3-71 administration (Tg-ANAVEX, P≤0.01). As shown in FIG. 3, Tg-sal rats showed less interaction time with a “stranger rat” than the wt-sal (P≤0.001) in the Social Preference test (SP). This deficit was recovered in the Tg-ANAVEX (P≤0.0001). As shown in FIG. 4, during the acquisition phase of the Morris Water Maze (MWM), the rats showed significant differences in learning in the last three days. Averaging escape latencies from day 3 to 5, the Tg-sal required more time to find the hidden platform than wt-sal (P≤0.01). Conversely, Tg-ANAVEX performed significantly better than the Tg-sal (P≤0.05). FIG. 5 shows that McSA1-immunoreactive (IR) plaques intensity of Tg-ANAVEX was significantly lower in CA1 (P≤0.01) and the cortex (P≤0.05) compared to the Tg-sal. This result confirmed that A3-71 prevented McGill-APP rats from increasing cortical and hippocampal extracellular Aβ deposition. FIG. 6 shows that Iba1-IR and GFAP-IR cells in Tg-ANAVEX were significantly less recruited in the proximity of the CA1 neurons compared to the Tg-sal (P≤0.05, for both). This result demonstrated that A3-71 reduced microglia and astrocytes recruitment towards Aß-burdened neurons in the hippocampus.

### Example 4. Conclusion

In summary, the results demonstrated the long-lasting effect of ANAVEX3-71 in preventing cognitive decline of McGill-R-Thy1-APP rats, even after a wash-out period. The results suggest preventative and prophylactic disease-modifying properties of ANAVEX3-71 over the AD-like amyloid pathology. The results also provide insights on the beneficial effects of M1 muscarinic and sigma-1 receptor agonists for neurodegenerative diseases involving amyloid pathology.

1. A method of prophylactic treatment of a degenerative disease manifested by amyloid pathology in a subject, the method comprising administering to the subject a prophylactically effective amount of a sigma-1 receptor agonist.

2-5. (canceled)

6. The method of claim 1, wherein the sigma-1 receptor agonist comprises ANAVEX2-73 (A2-73), ANAVEX 19-144, ANAVEX1-41, AV1066, ANAVEX3-71 (A3-71), PRE-084, Donepezil, Fluvoxamine, Amitriptyline, L-687,384, SA-4503, Dextromethorphan, Dimethyltryptamine, (+)-pentazocine, or any of their combinations, crystal forms, enantiomers and pharmaceutically acceptable salts thereof.

7. The method of claim 6, wherein the sigma-1 receptor agonist comprises a A3-71 amorphous form, a A3-71 crystal form, a A3-71 enantiomer, a A3-71 prodrug or a combination thereof.

8. The method of claim 1, wherein the amyloid pathology comprises amyloid formation, amyloid deposit, or amyloid plaque buildup.

9. The method of claim 1, wherein the degenerative disease comprises Alzheimer's disease, Parkinson's disease, Huntington's disease, Amyotrophic Lateral Sclerosis, Prion disease, or any combination thereof.

10. The method of claim 9, wherein the prophylactically effective amount is the amount to diminish or reduce the likelihood or seriousness of the degenerative disease.

11. The method of claim 9, wherein the prophylactically effective amount is the amount to substantially diminish the likelihood of the degenerative disease.

12. The method of claim 9, wherein the prophylactically effective amount is the amount to prevent the onset or deter the progress of the degenerative disease.

13. The method of claim 9, wherein the prophylactically effective amount is the amount to prevent memory loss and/or cognition decline cause by the degenerative disease.

14. The method of claim 9, wherein the prophylactically effective amount comprises about 0.1 mg to about 500 mg, preferably from about 5 mg to about 60 mg.

15. The method of claim 1, wherein the subject is a human subject or a non-human mammal.

16. A method of therapeutic or prophylactic treatment of a disease characterized by abnormal amyloid formation, amyloid plaques, plaque buildups or amyloid deposit in a subject comprising administering to the subject a therapeutically or prophylactically effective amount of a sigma-1 receptor agonist.

17-20. (canceled)

21. The method of claim 16, wherein the sigma-1 receptor agonist comprises ANAVEX2-73 (A2-73), ANAVEX 19-144, ANAVEX1-41, AV1066, ANAVEX3-71 (A3-71), PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof.

22. The method of claim 21, wherein the sigma-1 receptor agonist comprises a A3-71 amorphous form, a A3-71 crystal form, a A3-71 enantiomer, a A3-71 prodrug or a combination thereof.

23. The method of claim 16, wherein the disease is manifested by amyloid plaques or plaque buildups.

24. The method of claim 16 wherein the disease comprises Alzheimer's disease, Parkinson's disease, Huntington's disease, Amyotrophic Lateral Sclerosis, Prion disease, or any combination thereof.

25. The method of claim 24, wherein the therapeutically effective amount is the amount to diminish the likelihood or onset of the disease.

26. The method of claim 24, wherein the prophylactically effective amount is the amount to reduce the likelihood or seriousness of the disease.

27. The method of claim 24, wherein the prophylactically effective amount is the amount to prevent the onset or deter the progress of the disease.

28. The method of claim 24, wherein the prophylactically effective amount is the amount to prevent memory loss and/or cognition decline.

29. The method of claim 24, wherein the therapeutically or prophylactically effective amount comprises about 0.1 mg to about 500 mg, preferably from about 5 mg to about 60 mg.

30. The method of claim 16, wherein the subject is a human subject or a non-human mammal.

31-43. (canceled)
